(19) United States
(12) Patent Application Publication
(10) Pub. No.: US 2018/0369478 A1
(43) Pub. Date: Dec. 27, 2018

(54) MEDICAL INFUSION PUMPS AND SYSTEMS

(71) Applicant: Smiths Medical ASD, Inc., Plymouth, MN (US)

(72) Inventors: Michael KERSCH, St. Michael, MN (US); David DeBELSER, Plymouth, MN (US)

(73) Assignee: Smiths Medical ASD, Inc., Plymouth, MN (US)

(21) Appl. No.: 15/777,482

(22) PCT Filed: Nov. 4, 2016
(86) PCT No.: PCT/US2016/060625

§ 371 Date: May 18, 2018

Related U.S. Application Data
(60) Provisional application No. 62/256,982, filed on Nov. 18, 2015.

(51) Int. Cl. A61M 5/168; A61M 5/142; A61M 5/172; G16H 20/17

(52) U.S. Cl. CPC A61M 5/16877; A61M 5/142; A61M 5/1723; A61M 2205/505; A61M 2205/3303; A61M 2205/3334; G16H 20/17

(57) ABSTRACT

A medical infusion pump for delivering a medicament to a patient. The pump includes a user interface to display a choice of nominal medicament delivery rates. The user interface enables a practitioner to select one of the displayed nominal medicament delivery rates. It further enables the practitioner to enter a numerical multiplier factor, thereby creating a nominal delivery rate. It further enables the practitioner to create a custom alphanumeric string that the practitioner associates with a patient characteristic, enter a numeric value relating to the patient characteristic, and determine an actual delivery rate by modifying the selected nominal medicament delivery rate based on the numeric value relating to the patient characteristic. The pump mechanism is then controlled to deliver medicament from a reservoir to a patient at the actual delivery rate.

### CROSS-REFERENCE TO RELATED APPLICATIONS

The present application is a National Phase entry of PCT Application No. PCT/US2016/060625, filed on Nov. 4, 2016, which claims priority to U.S. Provisional Patent Application No. 62/256,982, filed on Nov. 18, 2015, which are hereby fully incorporated herein by reference.

### TECHNICAL FIELD

Subject matter of this disclosure relates generally to medical devices. More specifically, subject matter of this disclosure relates to medical infusion pumps and systems that are enabled to accept, and function with, practitioner-defined parameters that are in turn associated with selected patient characteristics or measurements.

### BACKGROUND

In an interest of reducing health care treatment costs, some medical infusion pumps may be designed with capabilities for treating multiple patient ailments. Such capabilities may be provided by relatively versatile medical infusion pumps that are able to deliver multiple medicament delivery protocols. A hospital may therefore advantageously avoid costs that would otherwise result from purchasing therapy-specific medical infusion pumps for every possible therapy. A hospital may also therefore advantageously limit a required inventory of medical infusion pumps, to more closely match an anticipated number of patients on particular medicament delivery protocols.

Analogously to oral medications such as pills and tablets that are provided in various names and concentrations, infusates that are delivered to patients by medical infusion pumps may also have various names and concentrations. Thus, medical infusion pumps may be programmed with menu choices listing medicament names, concentrations, and possible delivery rates. Some medical infusion pumps are therefore programmed by their manufacturers to display menus of choices of medicament delivery protocols that the particular pumps are capable of delivering. The medicament delivery protocol menus in some pumps are programmed by, for example, pharmacists who select pre-defined medicament delivery protocols from manufacturer-supplied computer programs.

As advances in medical science continue, there exists a need among medical professionals such as doctors, nurses, pharmacists, biomedical engineers, and other authorized users (collectively, “practitioners”) to be able to add experimental or patient-specific medicament delivery protocols to menus of medical infusion pumps in addition to protocols that are typically pre-programmed and/or those available for installation from manufacturer-supplied or -sponsored computer programs. In particular, there exists a need to enable flexibility of new medicament delivery protocols and/or therapies by enabling practitioners to use existing medical infusion pumps with new ratios and/or units of measurement on infusion pumps and/or approved preprogrammed delivery protocols.

### SUMMARY OF THE DISCLOSURE

Embodiments disclosed herein include medical infusion pumps and systems that are capable of delivering medicaments to patients. Medical infusion pumps and systems of these embodiments offer advantages of providing practitioners with extensive options for therapy-based programming while maintaining relatively simple and safe interfaces. At least one of the options, for therapy based programming can, for example, enable a practitioner to define customized medicament delivery protocols specific to new experimental and/or newly conceived medicament delivery protocols and/or therapies. Such customized medicament delivery protocols can represent entirely new protocols beyond what have been typically available from manufacturers, thereby providing the practitioner with extended pump functionality without, for example, a need for a pump manufacturer to add new ratios and/or units of measure each time a new medicament delivery protocol is conceived of.

In an embodiment, a medical infusion pump includes a pump mechanism, a memory, a user interface, and a processor operatively coupled with the pump mechanism, the memory, and the user interface. The processor can be configured and programmed to: enable the user interface to display a choice of medicament names, concentrations, and nominal delivery rates; enable the practitioner to create a custom alphanumeric string that the practitioner associates with a patient characteristic; enable the practitioner to enter a numerical value relating to the patient characteristic; determine an actual delivery rate by modifying the selected nominal medicament delivery rate based on the numeric value relating to the patient characteristic; and control the pump mechanism to cause medicament to be delivered from a reservoir to the patient at the actual delivery rate. A particular order of menus and choices presented to the practitioner may be configured or accessed in other sequences and combinations.

In an embodiment, the user interface may be a computing device that is not permanently connected to the pump. For example, a desktop computer, laptop computer, telephone, other computing device, or mobile device, can be used or serve as the user interface.

In embodiments, a practitioner may desire to begin pump programming by entering a custom or particular desired alphanumeric string and programming an algorithm into the medical infusion pump that is to run in conjunction with the alphanumeric string that the practitioner associates with the patient characteristic in the menu. The practitioner can continue programming by entering at least one of a medicament name, medicament concentration, and nominal delivery rate. The resulting medicament delivery protocol can be communicated to the pump to control its operation.

In one embodiment, the programmed algorithm running in conjunction with the alphanumeric string includes one or more numerical multiplier factors. Numerical multiplier factors may be entered into the medical infusion pump in relation to the patient characteristic being measured or observed. For example, in some embodiments, the programmed algorithm multiplies a numerical value relating to the patient characteristic with a numerical multiplier factor to produce an output product. In some embodiments, the output product corresponds to a medicament delivery rate. Accordingly, the programmed algorithm enables practitioners to program new or customized medicament delivery protocols and/or therapies beyond what have been typically available from manufacturers, thereby providing the practitioner with extended pump functionality.

The numerical multiplier factor may be entered using a user interface, or by causing the medical infusion pump to be in communication with a computer or another medical device. In some embodiments, the numerical multiplier factor may be a fixed number, such as a positive or negative integer or fraction, so as to produce a linear output product. In other embodiments, the numerical multiplier factor may be keyed to increase or decrease proportionately to the measured patient characteristic, so as to produce a non-linear output product. For example, the numerical multiplier factor could be a function of the numerical value relating to the patient characteristic.

In operation, the actual delivery rate of the medical infusion pump may be determined by: entering a fixed medicament delivery rate; selecting a preprogrammed medicament delivery protocol that adjusts the actual delivery rate according to a manufacturer or pharmacist defined medicament delivery protocol by creating a custom or particular desired alphanumeric string associated with an algorithm that adjusts the actual delivery rate as a function of a numerical value associated with a patient characteristic or a combination thereof. A particular order of menus and choices presented to the practitioner may be configured or accessed in other sequences and combinations.

Further disclosed herein is a system for controlling the rate of infusion delivery by a medical infusion pump. In this system, the medical infusion pump is programmable with an alphanumeric string associated with a patient characteristic. The memory of the medical infusion pump is configured to accept the algorithm associated with the alphanumeric string after manufacture. The algorithm may be associated with the patient characteristic after manufacture of the medical infusion pump. The medical infusion pump is further programmable with a numerical value relating to the patient characteristic. The medical infusion pump or remote computing device uses the algorithm to calculate an actual delivery rate as a function of the numerical value relating to the patient characteristic. The medical infusion pump is configured to communicate with a remote computing device or a sensor of a patient characteristic. The sensor of the patient characteristic is configured to communicate a signal to the medical infusion pump or the computer. The medical infusion pump is further configured to adjust the actual delivery rate of the pump mechanism in relation to the content of the signal received from the sensor or the computer, and according to an algorithm residing in the memory of the medical infusion pump.

The summary above is not intended to describe each illustrated embodiment or every implementation of the present disclosure and subject matter hereof. The figures and the detailed description that follow more particularly exemplify these embodiments.

### BRIEF DESCRIPTION OF THE DRAWINGS

Subject matter hereof may be more completely understood in consideration of the following detailed description of various embodiments in connection with the accompanying figures, in which:

FIG. 1 is a schematic diagram of a medical infusion pump, in accordance with an embodiment;

FIG. 2 is a flow diagram depicting the process flow for programming a medicament delivery protocol according to an embodiment;

FIG. 3 is a schematic diagram depicting a practitioner performing one or more portions of a medicament delivery protocol according to an embodiment through a user interface not physically coupled to a medical infusion pump;

FIG. 4 us a flow diagram depicting a process flow for programming a custom medicament delivery protocol according to an embodiment; and

FIG. 5 is a schematic diagram depicting a system in which a sensor of a patient characteristic is in communication with a medical infusion pump, and/or a practitioner's device that is device in communication with the pump.

While embodiments of the present disclosure and subject matter hereof are amenable to various modifications and alternative forms, specifics thereof are shown by way of example in the drawings and will be described in detail. It should be understood, however, that the intention is not to limit the disclosure and/or subject matter hereof to the particular embodiments described. On the contrary, the intention is to cover all modifications, equivalents, and alternatives falling within the spirit and scope of the disclosure and/or subject matter hereof as defined by the appended claims.

### DETAILED DESCRIPTION

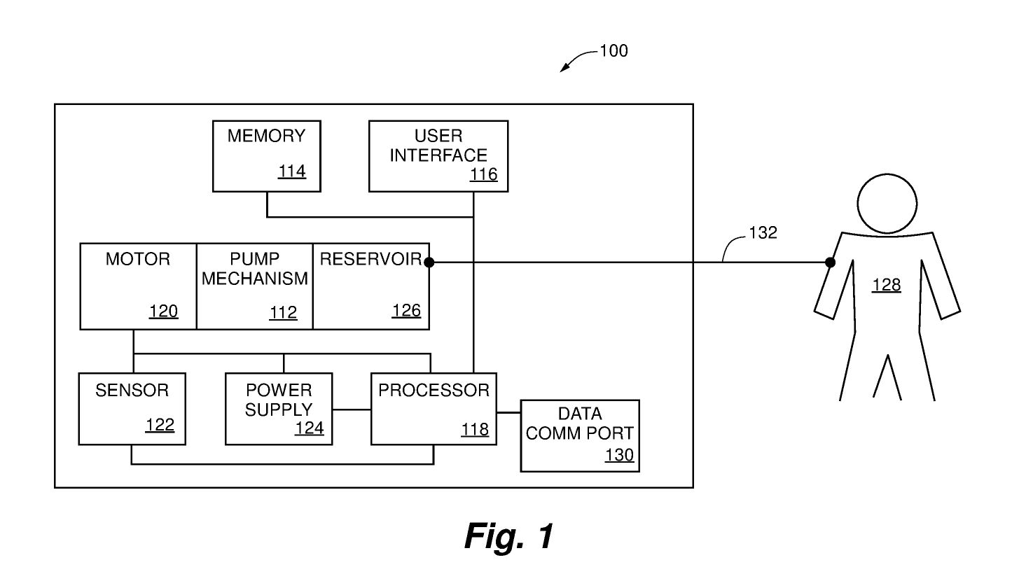

With reference to FIG. 1, a medical infusion pump 100 can comprise a pump mechanism 112, a memory 114, a user interface 116, and a processor 118 operatively coupled with the pump mechanism 112, the memory 114, and the user interface 116. It is to be appreciated and understood that pump 100 may be any suitable medical infusion pump such as, for example, a so-called ambulatory pump, a large volume pump, a patient-controlled analgesia (PCA) pump, an elastomeric pump, a syringe pump, an enteral pump, or an insulin pump. For example, in an embodiment, pump 100 can be a CADD®-Solis Ambulatory Infusion Pump, a CADD®-Solis Variable Infusion Profile (VIP) Ambulatory Infusion Pump, a Medfusion® 4000 syringe pump, or other pump running clinical software, such as the Pharm Guard Administrator Medication Safety Software (MSS).

Pump mechanism 112 can be driven by a motor 120. Operation of motor 120 can be controlled, at least in part, by a sensor 122. Pump 100 can receive operational power through a power supply 124. Pump mechanism 112 is operatively coupled to a reservoir 126 to controllably deliver medicament contained therein to a patient 128. Reservoir 126 can be enclosed entirely within medical infusion pump 100, partially within pump 100, mounted to pump 100, or remote from pump 100. Medical infusion pump 100 may optionally include at least one data communication port 130 for receiving data by wired or wireless connection.

Delivery of the medicament to patient 128 can occur transdermally or through a fluid passageway 132 in fluid communication with the patient 128. Such a fluid passageway 132 can connect to patient 128 through, for example, the patient's skin, intravenously, or via a gas that is directed to and enters the patient's respiratory system.

Processor 118 can be configured and programmed to enable user interface 116 to display, for example, a set of medicament names, concentrations, and nominal or typical delivery rates to be selected by a practitioner. In an embodiment, user interface 116 is configured to enable a practitioner to enter a Patient Specific Pump Parameter (PSPP), or a plurality of PSPPs, that directly affect the amount of medicament delivered to the patient and can include hard and soft programming limits. For example, in an embodiment a PSPP is adjusted to the patient's body weight. In embodiments, quantity or concentration of a medicament can be defined by Base Units (BaU), which can be set or adjusted by a practitioner. For example, BaU can be in units of mL, mg, and mcg. Base Units can also represent PSPP delivery amounts per some unit of time (rates in Base Units per time; e.g. BaU/hr). In some embodiment, processor 118 can further be loaded with Therapy Units (ThU), which are open-ended user configurable option-enabled text strings that enable one or more BaUs and/or PSPPs to be extended into therapy specific units of measure (e.g., BaU/ThU per time unit). The following Table 1 lists examples of User-Configured Therapy Units.

TABLE 1

| | User | | | |
|---|---|---|---|---|
| | configured | User configured | Continuous Rate | User defined meaning of |
| Therapy | Therapy Units | Therapy Unit Amount | units of measure | Therapy Units |
| Weight | “kg” | 250 kg | ml/kg/hr | Kilograms |
| Height | “cm” | 125 cm | mcg/cm/hr | Centimeters |
| Body Surface Area | “L × W” | 2000 L × W | ml/L × W/hr | Length × Width |
| Body Tissue Volume | “cc” | 100 cc | mg/cc/hr | Cubic Centimeters |
| Fluid Intake | “oz” | 32 oz | mg/oz/hr | Amount of Ingested |
| | | | | Fluids per Day |

Other suitable therapy specific units of measure are also contemplated. For example, processor 118 can be configured and programmed with any suitable therapy specific units of measure based on any observable and/or measureable patient characteristic correlatable to infusion therapy.

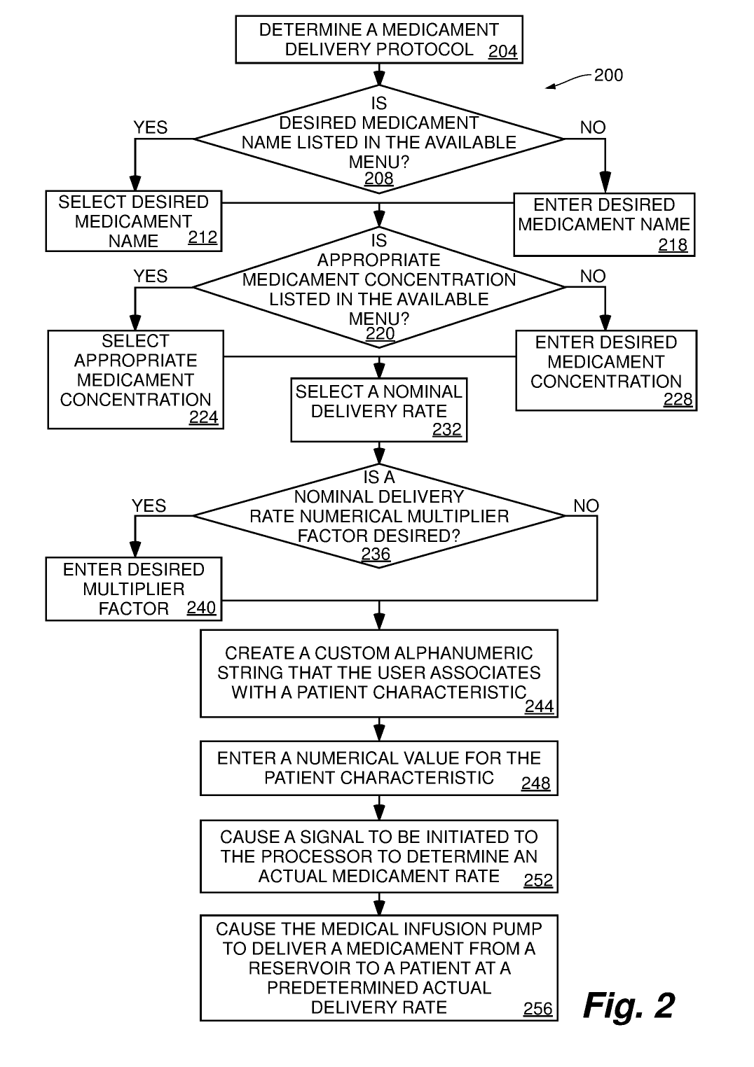

Referring to FIG. 2, in an example embodiment of the subject matter hereof, a process 200 of programming medical infusion pump 100 can begin with a determination by a practitioner of a desired medicament delivery protocol to cause pump 100 to deliver medicament from pump 100, as indicated at step 204. At step 208, the practitioner determines whether a desired medicament name is listed in a menu displayed on user interface 116 of pump 100. If the desired medicament is so listed, then at step 212 the practitioner selects that medicament from a menu of medicaments displayed on user interface 116. If the desired medicament name is not listed in the menu displayed on user interface 116, then at step 218 the practitioner manually enters the desired medicament name.

At step 220, the practitioner determines whether a desired medicament concentration is listed in a menu displayed on user interface 116 of pump 100. If the desired concentration is so listed, then at step 224 the practitioner selects that concentration from a menu of concentrations displayed on user interface 116. If the desired medicament concentration is not listed in the menu displayed on user interface 116, then at step 228 the practitioner manually enters the desired medicament concentration.

It is to be understood that menu choices in steps 208 and 220 may be combined into a single step. For example, the menu can be programmed such that the menu displayed on user interface 116 provides a list of combinations of both medicament names and concentrations, thus shortening the process but potentially also increasing the number of choices in the menu displayed on user interface 116.

At step 232, the practitioner selects a nominal medicament delivery rate in the menu displayed on user interface 116. After the selection of a nominal medicament delivery rate has been made, then at step 236 the practitioner determines whether a nominal medicament delivery rate multiplier factor is desired. If a nominal medicament delivery rate is desired, then at step 240 the desired multiplier factor may be entered. In other embodiments, the desired multiplier factor is entered in conjunction with step 244.

It is also to be understood that the choices in steps 232 and 236 may occur in reverse order. A selection of either of the nominal delivery rate or the nominal medicament rate multiplier factor can occur before the other.

At step 244, the practitioner creates a custom or particular desired alphanumeric string that the practitioner associates with a patient characteristic and enters it into user interface 116. At step 244 the practitioner may also enter an algorithm associated with the alphanumeric string. In some embodiments, the algorithm is configured to determine the nominal and/or actual medicament delivery rate. The algorithm can include one or more numerical multiplier factors for multiplication with a numerical value relating to a patient characteristic to produce a product output for use in determining an actual medicament delivery rate. It is to be understood that the sequence of the process flow indicated at steps 208, 220, 232, 236, and 244 can be presented to the practitioner in a variety of different orders without departing from the spirit and scope of this disclosure or the subject matter hereof.

At step 248, the practitioner enters a numerical value relating to the patient characteristic. At 252, the practitioner causes a signal to be initiated to processor 118 to determine an actual medicament delivery rate, wherein the entered numerical value relating to the patient characteristic is a factor in determining the actual delivery rate. The actual delivery rate may be stored in memory 114, or be communicated to a remote memory through data communication port 130. At step 256, pump 100 delivers medicament from reservoir 126 to patient 128 at a predetermined actual delivery rate.

A particular order of menus and choices displayed to the practitioner may be configured or accessed in other sequences and combinations. It is further to be appreciated that user interface 116 may be physically coupled, or not physically coupled, to medical infusion pump 100 and can be remote from pump 100 but remain in data communication with pump 100.

Further with respect to the example process flow illustrated in FIG. 2, in some embodiments user interface 116 of pump 100 provides, at step 232, choices of nominal medicament delivery rates in, for example, units of weight per time or volume per time. Examples of nominal medicament delivery rates include, but are not limited to, units of micrograms, milligrams, grams, or other units of weight; microliters, milliliters, liters, or other units of volume; and per second, per minute, per hour, per day, per therapy duration, or other units of time.

The alphanumeric string that a practitioner associates with a patient characteristic at step 244 is added to the menu of medical infusion pump 100 through user interface 116. Examples of user interfaces include, but are not limited to, a keypad, a touch screen, a pointer, a sensor of non-tactile human interaction, or a practitioner's device such as a computer or a mobile telephone. User interface 116 may be in direct communication with pump 100, or indirectly coupled to the pump 100 through a wireless network, hospital information system, mobile telephone network, satellite connection, or other technique of transmitting data from one device to another.

The patient characteristic may be an objectively measured or subjectively observed characteristic. Examples of patient characteristics include, but are not limited to, weight, height, girth, body surface area, body tissue volume, body mass index, fluid intake, body temperature, blood carbon dioxide saturation, blood glucose level, heart rate, respiration rate, blood pressure, cholesterol level, red blood cell count, white blood cell count, blood acidity, urine acidity, breath composition, age, gender, DNA profile, parasite burden, brainwave activity, or the patient's condition as perceived by one or more of a practitioner's senses.

In an embodiment of medical infusion pumps and systems that are enabled to accept, and function with, practitioner-defined parameters that are in turn associated with selected patient characteristics or measurements—as described by example with reference to FIGS. 1 and 2 or otherwise contemplated with reference to any of the illustrations herein—it is to be appreciated and understood that a specific application of subject matter hereof can improve the function and efficiency of operation for practitioners of infusion pumps and systems. For example, in the system of FIGS. 1 and 2, pump 100 can advantageously be controlled by the system to deliver a medicament at 256 from pump 100 (and associated reservoir 126) to patient 128 at a predetermined actual delivery rate. In particular, the aforedescribed process 200 of programming medical infusion pump 100 can cause pump mechanism 112, with memory 114, user interface 116, and processor 118 operatively coupled thereto, to at least in part command operation of motor 120 in cooperation with sensor 122 and thereby controllably deliver medicament contained in reservoir 126 to patient 128.

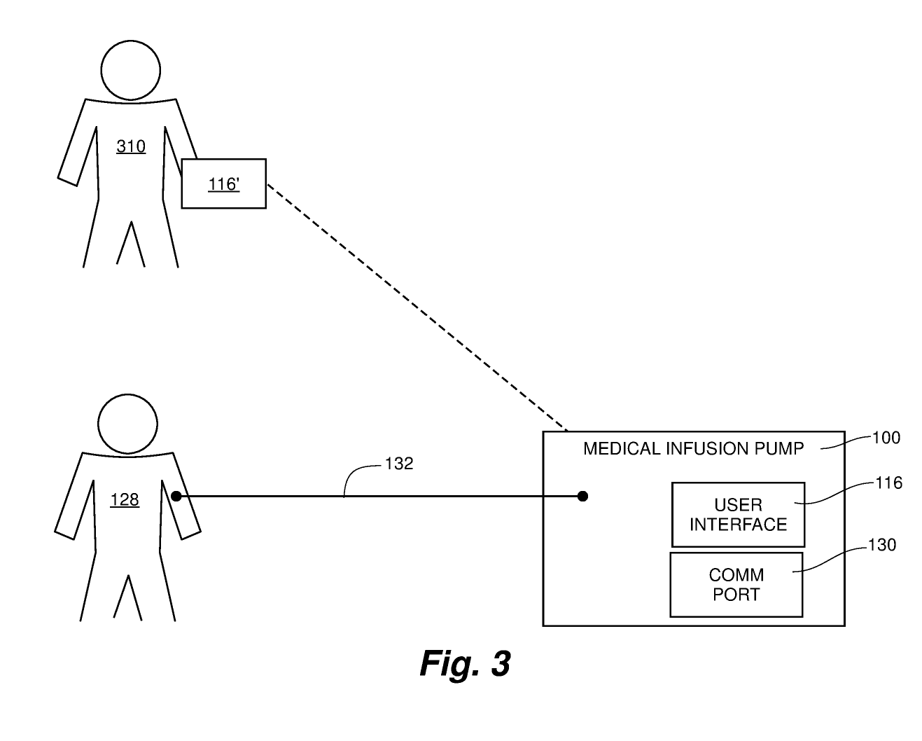

As illustrated in FIG. 3, a practitioner 310 may desire to program, using a remote user interface 116′, an algorithm into medical infusion pump 100 that is to run in conjunction with the alphanumeric string that the practitioner associates with the patient characteristic at step 244 in FIG. 2 to effect the actual medication delivery rate to patient 128 (via any of the aforementioned routes such as transdermally or through fluid passageway 132). It is to be particularly appreciated and understood that, advantageously, such programming of pump 100 can occur after pump 100 has been manufactured pursuant to novel and inventive subject matter hereof—as described by example or otherwise contemplated throughout this document and accompanying drawings.

The algorithm can be used to create a linear or nonlinear relationship of medicament delivery rate to a particular patient characteristic. For example, if the measured patient characteristic is urine output over a period of time, the algorithm associated with urine output might responsively cause a proportionate rate of hydration fluid to be infused by pump 100. In another example, if the measured patient characteristic is body temperature, the algorithm associated with a medicament intended to raise body temperature might responsively cause a decrease in delivery rate of the medicament as the body temperature of the patient approaches a target value. In another example, if the measured patient characteristic is a characteristic known or believed to exhibit a delayed response to medicine, such as a blood glucose level reaction to insulin, the algorithm associated with diabetes could responsively cause an intermittent bolus pattern of delivery from pump 100 (e.g., varying bolus delivery rates and times). In yet another example, if the measured patient characteristic is brainwave activity, the algorithm associated with a medicament might responsively cause the delivery of anesthesia, or other medicament, to be altered in response to the measured brainwave activity.

In another embodiment, the measured patient characteristic could be blood pressure, or a cyclical patient characteristic, such as heart rate, breathing rate, brainwave activity, and so forth. Therefore, algorithms associated with these patient characteristics could result in cyclical medicament delivery rates that change in response to the measured patient characteristic. In yet another embodiment, subjectively observed patient characteristics, such as flushness of skin, pupil dilation, patient complaints of inability to urinate, verbal expressions of erratic thought, slurred speech, sweating, and so forth, can be associated with algorithms to responsively adjust medicament delivery rates.

Numerical values may be entered into medical infusion pump 100 in relation to the patient characteristic being measured or observed. The numerical value may be entered using user interface 116, or by causing pump 100 to be in communication via data communication port 130 with user interface 116 of a device not physically connected to infusion pump 100 as aforedescribed. The device may be in direct communication with pump 100 or may communicate with pump 100 through an intermediate data transmission component such as a server or wireless router.

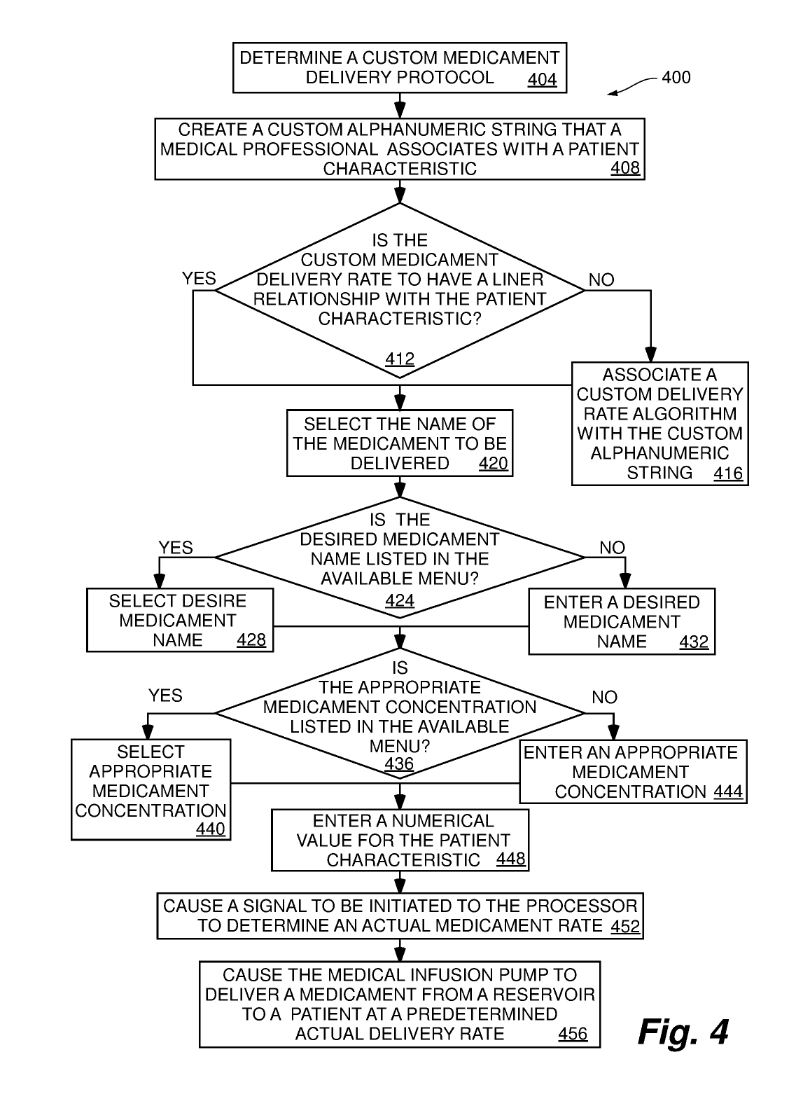

With reference to FIG. 4, in an example embodiment of the novel and inventive subject matter hereof, a process 400 of programming medical infusion pump 100 with a custom medicament delivery protocol begins at step 404, at which a custom medicament delivery protocol is determined. At step 408, a practitioner creates a custom alphanumeric string that the practitioner associates with a patient characteristic.

At step 412, the practitioner decides whether the custom or particular desired medicament delivery rate is to be in linear relationship to the patient characteristic. If the delivery rate is to be in such linear relationship, then at step 420 a name of the medicament to be delivered is selected on user interface 116 or 116′. If the custom or particular desired delivery rate is to not be in such linear relationship, then at step 416 a desired delivery rate algorithm is associated with the desired alphanumeric string, for example, by way of user interface 116 or 116′. In some embodiments, the algorithm is configured to determine the medicament delivery rate. For example, the algorithm can include one or more numerical multiplier factors for multiplication with a numerical value relating to a patient characteristic to produce a product output for use in determining an actual medicament delivery rate. In other embodiments, the numerical multiplier factor can be a function of the numerical value relating to the patient characteristic to produce a product output for use in determining an actual medicament delivery rate.

At step 420, the name of the medicament to be delivered is selected by using user interface 116 or 116′. At step 424, the practitioner determines whether the desired medicament name is listed in a menu of names that is displayed on user interface 116 or 116′. If the desired medicament name is so listed, then at step 428 the name is selected from the menu. If the desired name is not so listed, then at step 432 the name is entered using user interface 116 or 116′.

At step 436, the practitioner determines whether a desired medicament concentration is listed in a menu displayed on user interface 116 or 116′. If the desired concentration is so listed, then at step 440 the practitioner selects that concentration from a menu of concentrations displayed on user interface 116 or 116′. If the desired medicament concentration is not listed in the menu displayed on user interface 116 or 116′, then at step 444 the practitioner manually enters the desired medicament concentration.

It is to be understood that menu choices presented at steps 424 and 436 may be combined into a single step. For example, the menu can be programmed such that the menu displayed on user interface 116 or 116′ provides a list of combinations of both medicament names and concentrations, thus shortening the process but potentially also increasing the number of choices in the menu displayed on user interface 116 or 116′.

At step 448, the practitioner uses either user interface 116 or mobile user interface 116′ to enter a numerical value relating to the patient characteristic to cause, at step 452, processor 118 to determine an actual medicament delivery rate. In one embodiment, the actual medicament delivery rate is determined according to the delivery rate algorithm associated with the desired alphanumeric string at step 416, wherein the entered numerical value relating to the patient characteristic is a factor in determining the actual medicament delivery rate determined according to the algorithm. At step 456, medical infusion pump 100 delivers medicament from reservoir 126 at a predetermined delivery rate.

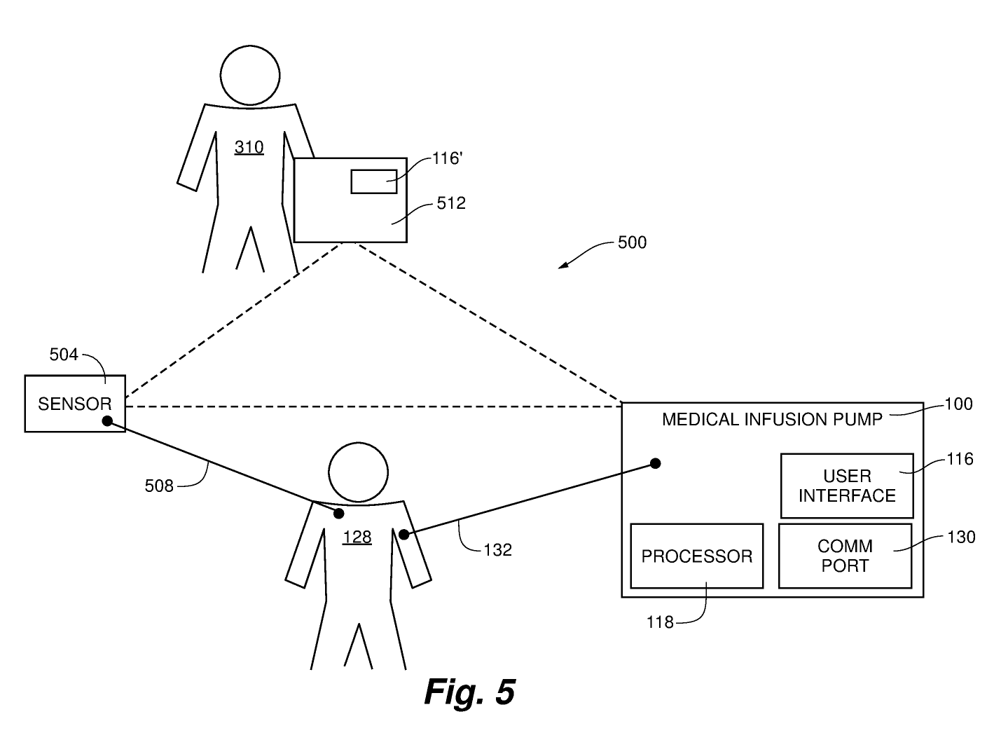

With reference to FIG. 5, a system 500 can be configured to control a rate of infusion delivery by a medical infusion pump. A medical infusion pump such as pump 100 that has been programmed as described and illustrated herein with an alphanumeric string that a practitioner associates with a patient characteristic, and that has been further programmed with a numerical value relating to the patient characteristic, and whose processor 118 calculates an actual delivery rate based on the alphanumeric string and numerical value relating to the patient characteristic, can be configured to deliver medicament at an actual medicament rate to patient 128 transdermally or through fluid passageway 132.

A sensor 504 of a patient characteristic may receive patient characteristic information by communication path 508. Communication path 508 may be tailored to the characteristic to be sensed and communicated. For example, if the characteristic to be communicated is weight, height, or girth, patient 128 may be in operable and/or physical contact with the sensor. In another example, if the characteristic is body surface area, patient 128 may be in contact with the sensor via a body scanning device. In yet another example, if the characteristic is body tissue volume, patient 128 may be in contact with fluid in a fluid displacement chamber. In another example, if the characteristic is body mass index, patient 128 may be in operable and/or physical contact with a caliper device. In a further example, if the characteristic is fluid intake, patient 128 may be in contact with a fluid delivery system that is in communication with a sensor. In even more examples, patient 128 may be in operable and/or physical contact with a sensor of body temperature, blood carbon dioxide saturation, blood glucose level, heart rate, respiration rate, blood pressure, cholesterol level, red blood cell count, white blood cell count, blood acidity, urine acidity, breath composition, age, gender, DNA profile, parasite burden, brainwave activity, and so forth.

Pump 100 is configured to communicate with a practitioner's device 512 (such as, for example a remote computer) and a sensor 504 of a patient characteristic. Sensor 504 of the patient characteristic is configured to communicate a signal to medical infusion pump 100 and/or device 512. Pump 100 is further configured to adjust the actual delivery rate of pump mechanism 112 (as shown in FIG. 1) in relation to content of the signal received from sensor 504 and/or device 512, according to an algorithm residing in memory 114 of pump 100. Memory 114 of pump 100 can be configured to accept a custom or desired particular delivery rate algorithm after manufacture. The custom or desired particular delivery rate algorithm can be associated with the patient characteristic after manufacture of the medical infusion pump.

In an embodiment, device 512 can be loaded with manufacturer-provided software that enables a computer program to accept a custom delivery rate algorithm after the manufacturer provided the software. In some embodiments, the algorithm can be tailored to be used in conjunction with an experimental new delivery protocol and/or therapies and communicated to pump 100, thereby enabling use of pump 100 beyond the constraints imposed by the FDA on infusion pumps and/or approved preprogrammed delivery protocols as aforementioned. Irrespective of a particular embodiment, it is to be appreciated and understood that novel and inventive subject matter hereof—as described by example or otherwise contemplated herein—could, for example, advantageously enable or assist infusion therapy research. In particular, such an infusion pump system could be used rather easily and efficiently in investigating relationships between medicament delivery from a pump in various therapy units and some externally-monitored patient physiological state or states based on feedback from a patient monitoring or other device in communication with the pump system.

In an embodiment, device 512 may be a desktop computer, laptop computer, telephone, mobile computing device, or other wired or wireless device capable of satisfactory performance, and so forth. In an embodiment, sensor 504 can be a practitioner who is trained to perceive, with that practitioner's own senses, a condition or conditions of patient 128 and operate a desktop computer, laptop computer, telephone, mobile computing device, or other wired or wireless device capable of satisfactory performance, to communicate with pump 100 and/or device 512.

Irrespective of a particular embodiment, it is to be appreciated and understood that the novel and inventive subject matter hereof—as described by example or otherwise contemplated herein—advantageously provides an infusion pump system that allows a practitioner to program delivery protocols in user configurable “Therapy Units” that are specific to a particular infusion therapy for a particular patient. Thus, use of such an infusion pump system advantageously obviates deployment of many separate pumps that would otherwise be required for each of the many and various (and, possibly, uncommon or unusual) infusion therapy units that may be needed in treatment of various patients. It is also to be appreciated and understood that such an infusion pump system thereby advantageously provides practitioners with extensive options for more therapy-based programming, while keeping pump programming interfaces relatively simple with respect to myriad programming options thereby available to them.

Persons of ordinary skill in arts relevant to this disclosure and subject matter hereof will recognize that embodiments may comprise fewer features than illustrated in any individual embodiment described by example or otherwise contemplated herein. Embodiments described herein are not meant to be an exhaustive presentation of ways in which various features may be combined and/or arranged. Accordingly, the embodiments are not mutually exclusive combinations of features; rather, embodiments can comprise a combination of different individual features selected from different individual embodiments, as understood by persons of ordinary skill in the relevant arts. Moreover, elements described with respect to one embodiment can be implemented in other embodiments even when not described in such embodiments unless otherwise noted. Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended. Furthermore, it is intended also to include features of a claim in any other independent claim even if this claim is not directly made dependent to the independent claim.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of Section 112, sixth paragraph of 35 U.S.C. are not to be invoked unless the specific terms “means for” or “step for” are recited in a claim.

1. A medical infusion pump for delivering a medicament to a patient, the medical infusion pump comprising:
a pump mechanism;
a memory;
a user interface; and
a processor operatively coupled with the pump mechanism, the memory, and the user interface, the processor configured and programmed to:
enable a practitioner to select at least one of a medicament name and concentration from at least one displayed medicament name and concentration, or a manually entered medicament name and concentration;
enable the practitioner to select a nominal medicament delivery rate from at least one displayed nominal medicament delivery rate;
enable the practitioner to enter a numerical multiplier factor to adjust the nominal medicament delivery rate;
enable the practitioner to create a custom alphanumeric string associated with a patient characteristic;
enable the practitioner to enter a numerical value relating to the patient characteristic;
determine an actual medicament delivery rate by modifying the nominal medicament delivery rate based on a numerical multiplier factor of the numeric value relating to the patient characteristic; and
control the pump mechanism to deliver from a reservoir the medicament to the patient at the actual medicament delivery rate.

2. The medical infusion pump of claim 1, wherein the nominal medicament delivery rate is in at least one of units of weight per time and volume per time.

3. The medical infusion pump of claim 2, wherein the nominal medicament delivery rate is in at least one of: (i) units of micrograms, milligrams, grams, and other units of weight; (ii) units of microliters, milliliters, liters, and other units of volume; and (iii) units of per second, minute, hour, day, therapy duration, and other units of time.

4. The medical infusion pump of claim 1, wherein the patient characteristic is at least one of an objectively measured characteristic and subjectively observed characteristic.

5. The medical infusion pump of claim 4, wherein the patient characteristic represents at least one of the patient's weight, height, girth, body surface area, body tissue volume, body mass index, fluid intake, body temperature, blood carbon dioxide saturation, blood glucose level, heart rate, respiration rate, blood pressure, cholesterol level, red blood cell count, white blood cell count, blood acidity, urine acidity, breath composition, age, gender, DNA profile, brainwave activity, parasite burden, and the patient's condition as perceived by at least one of a practitioner's senses.

6. The medical infusion pump of claim 1, wherein the alphanumeric string is created using at least one of a keypad, a touch screen, a pointer, a sensor of non-tactile human interaction, and a computer.

7. The medical infusion pump of claim 1, wherein an algorithm residing in the memory is associated with the patient characteristic is at least one of a linear, non-linear and a combination thereof.

8. The medical infusion pump of claim 1, wherein the numerical value relating to the patient characteristic is entered using at least one of the user interface, a computer in communication with the medical infusion pump and a second medical device in communication with the medical infusion pump.

9. The medical infusion pump of claim 8, wherein the second medical device is at least one of a weight scale, a body temperature monitor, a pulse oximeter, an EEG, a continuous blood glucose monitor, a heart rate monitor, and a respiration rate monitor.

10. The medical infusion pump of claim 8, wherein the computer is at least one of a portable device in direct communication with the medical infusion pump and a remote computer in communication with the medical infusion pump through a hospital information management system.

11.-12. (canceled)

13. A system for controlling the rate of infusion delivery to a patient comprising:
a medical infusion pump, a computer and a sensor of a patient characteristic, the medical infusion pump including—
a pump mechanism;
a memory;
a user interface; and
a processor operatively coupled with the pump mechanism, the memory, and the user interface,
wherein the medical infusion pump is programmed with an alphanumeric string associated with a patient characteristic,
wherein the medical infusion pump is programmed with a numerical value relating to the patient characteristic,
wherein the medical infusion pump calculates an actual delivery rate based on the algorithm relating to the numerical value relating to the patient characteristic, and
wherein the medical infusion pump is configured to communicate with a computer,
the sensor of a patient characteristic being configured to communicate a signal to the medical infusion pump, and
wherein the medical infusion pump being configured to adjust the speed of the pump mechanism in relation to the signal received from the sensor according to an algorithm residing in the memory of the medical infusion pump,
the memory of the medical infusion pump is configured to accept the algorithm after manufacture, and
the algorithm is associated with the patient characteristic after manufacture of the medical infusion pump.

14. The system of claim 13, wherein the sensor of the patient characteristic is further configured to communicate a signal to a computer, wherein the computer is at least one of a desktop computer, a laptop computer, a telephone, and a mobile device, a wired device capable of satisfactory performance with the system for controlling the rate of infusion delivery by a medical infusion pump, and a wireless device capable of satisfactory performance with the system for controlling the rate of infusion delivery by a medical infusion pump.

15. The system of claim 13, wherein the sensor of a patient characteristic senses at least one of a patient's weight, height, girth, body surface area, body tissue volume, body mass index, fluid intake, body temperature, blood carbon dioxide saturation, blood glucose level, heart rate, respiration rate, blood pressure, cholesterol level, red blood cell count, white blood cell count, blood acidity, urine acidity, breath composition, age, gender, DNA profile, brainwave activity, and parasite burden.

16. The system of claim 13, wherein the sensor is a practitioner perceiving the condition of patient, wherein the signal is communicated to the medical infusion pump by at least one of a desktop computer, laptop computer, telephone, and mobile computing device, a wired device capable of satisfactory performance with the system for controlling the rate of infusion delivery by a medical infusion pump, and a wireless device capable of satisfactory performance with the system for controlling the rate of infusion delivery by a medical infusion pump.

17. A method for causing a medical infusion pump to deliver medicament to a patient, compromising:
determining a custom medicament delivery protocol;
creating a custom alphanumeric string associated with a patient characteristic;
entering a medicament by at least one of selecting the medicament from a preprogrammed menu and manually entering the medicament;
entering a nominal medicament delivery rate by at least one of selecting the nominal medicament delivery rate from a preprogrammed menu and manually entering the nominal medicament delivery rate;
entering a numeric value relating to the patient characteristic by at least one of manual entry and receiving a signal from a sensor of a patient characteristic;
causing the signal to be communicated to a processor to determine an actual medicament delivery rate; and
causing the medical infusion pump to deliver medicament from the reservoir to a patient at the determined actual delivery rate.

18. The method of claim 17, further comprising associating a custom delivery rate modifying algorithm with the custom alphanumeric string.

19. The method of claim 17, further comprising entering a numerical nominal medicament delivery rate multiplier factor.

20. The method of claim 17, further comprising programming the custom medicament delivery protocol remotely from the medical infusion pump, and communicating the protocol to to the medical infusion pump.
